(19) United States
(12) Patent Application Publication
(10) Pub. No.: US 2020/0238052 A1
(43) Pub. Date: Jul. 30, 2020

(54) CATHETER PORT

(71) Applicant: Rabie Stephan, Amherst, NY (US)

(72) Inventor: Rabie Stephan, Amherst, NY (US)

(21) Appl. No.: 16/830,828

(22) Filed: Mar. 26, 2020

Related U.S. Application Data
(63) Continuation-in-part of application No. 14/874,172, filed on Oct. 2, 2015, now Pat. No. 10,632,284.

(51) Int. Cl. A61M 25/01; A61M 25/02; A61M 39/02

(52) U.S. Cl. CPC A61M 25/0111; A61M 25/02; A61M 39/0247; A61M 2039/0273; A61M 2025/0266; A61M 2039/027; A61M 2205/0205; A61M 2025/0213

(57) ABSTRACT

A catheter port adapted to be selectively secured to a patient's scalp about a site at which a catheter is to be selectively inserted into, and removed from of the patient's head. The port includes a cushion adapted to be secured to the patient's scalp at its bottom surface about the site and at its top surface to a member, an inner tube having one portion mounted on the member and having another portion that is adapted to extend into the patient's head about the site for guiding movement of a catheter relative to the site and for reducing exposure of the catheter from the patient's scalp, an outer tube having a proximal end mounted on one of the member and inner tube, an intermediate portion extending away from the one of the member and the inner tube in a distal end and a septum mounted on the outer tube.

### PRIORITY CLAIM AND RELATED APPLICATIONS

This continuation-in-part application claims the benefit of priority from non-provisional application U.S. Ser. No. 14/874,172 filed Oct. 2, 2015. Said application is incorporated by reference in its entirety.

### BACKGROUND OF THE INVENTION

### 1. The Field of the Invention

The present invention is directed generally to a catheter port. More specifically, the present invention is directed to a catheter port adapted to facilitate the use of a catheter on a patient and isolate the catheter from the patient's skin or scalp.

### 2. Background Art

Numerous solutions have been attempted in combating infections caused by intravascularly implanted medical devices or catheters. The treatment of entry sites of percutaneously implanted devices have dramatically reduced infections caused by the transfer of microbes from the surface tissue or skin of a person or animal to the intravascular, organ or body tissues. However, infections due to microbes already present within the body remain serious cause of infections and ill health. Common solutions include, but not limited to, coating of surfaces with anti-microbial drugs, modification of surface charges, implementation of ultrasonic vibrating devices, etc.

However, such solutions may cause negative side effects such as in the case of anti-microbial drugs. Modification of surface charges and implementation of ultrasonic vibrating devices require complicated equipment which not only increase costs but also require constant or periodic power use, sophisticated control and actuating devices.

Catheter entry sites are fertile grounds for infectious agents, e.g., bacteria, fungi and viruses to be introduced into a patient's body by direct contact of catheters with unclean site surfaces, such as, the skin layer and percutaneous tissue. As a catheter is inserted into a patient's body, any infectious agents garnered from such surfaces can be drawn into the body quite readily. The Applicant discovered that a major source of infection is derived from the patient's skin layer and percutaneous tissue. By isolating such surfaces from the catheters, such infectious agents can be blocked from entering the patient's body. None of the references below discloses a catheter apparatus or device capable of preventing intrusion of infectious agents through catheter entry sites.

U.S. Pat. No. 5,817,072 to Lampropoulos et al. (hereinafter Lampropoulos) discloses an indwelling catheter apparatus for providing fluid to the central venous system. The indwelling catheter apparatus includes an indwelling cannula adapted for insertion into subcutaneous tissue in the chest wall and approximately within the superior vena cava, the indwelling cannula having an indwelling distal end, the distal end having an exit port in fluid communication with the superior vena cava; a proximal hub end adapted for positioning outside of the body; and a primary lumen for delivering fluid to the circulatory system; and delivery means for delivering fluid medicament to the subcutaneous tissue surrounding a portion of the cannula. Lampropoulos fails to disclose a mechanism for preventing intrusion of infectious agents through the punctured site through which the indwelling catheter apparatus is administered. Upon administering the apparatus, the Lampropoulos apparatus lacks a physical barrier that prevents infectious agents from being drawn into or from entering the punctured site. As the cylindrical wall (36 of Lampropoulos) is not secured against the skin layer or subcutaneous tissue, the catheter device (12 of Lampropoulos) is free to move with respect to such body parts. Lampropoulos also lacks a means for cushioning the catheter device (12 of Lampropoulos) against the surface upon which it is attached.

U.S. Pat. Pub. No. 20070282271 to Kaplan et al. (hereinafter Kaplan) discloses a device for protecting the proximal extension of an indwelling catheter and its percutaneous puncture site from moisture, fluids and risk of infection and maintains a clean environment and a fluid-impermeable and bacteria growth-inhibiting seal against the patient's skin when bathing, showering or swimming, and also protecting others from being exposed to the patient's blood and catheter at that time. A faceplate is adhered to the skin about the catheter exit site with medical adhesive, the proximal end of the catheter is secured to the faceplate to prevent it from moving from within the patient's body, and a water-impermeable housing is sealed to the faceplate, with the proximal end of the catheter extending into a cavity of the housing. An antimicrobial material may be disposed on the backside of the faceplate. The device allows a patient to shower, bathe and swim while preventing the catheter from being dislodged from the patient. Kaplan fails to disclose a mechanism for preventing intrusion of infectious agents through the catheter exit. Catheters can come in contact with the skin layer and subcutaneous tissue as the faceplate does not prevent such contact.

Thus, there is a need for a catheter port capable of isolating a catheter from contacting a patient's skin or scalp when the catheter is being administered to the patient or when the catheter is left in place upon its administration.

### SUMMARY OF THE INVENTION

In accordance with the present invention, there is provided a catheter port adapted to be selectively secured topically to a patient's scalp about a site at which a catheter is to be selectively inserted into, and removed from of the patient's scalp, the catheter port including:
a cushion adapted to be secured to the patient's scalp about the site, the cushion having a bottom surface arranged to face toward the patient's scalp, an aperture through which the catheter is to be disposed and a top surface attached to a first end of a member;
an inner tube having an inner diameter, a first end and a free second end, the inner tube mounted at the first end of the inner tube to the first end of the member, the free second end of the inner tube is adapted to only extend into the patient's head at and below the scalp about the site for guiding movement of the catheter relative to the site and for reducing exposure of the catheter from the patient's scalp; an outer tube having an inner diameter, a proximal end, an intermediate portion and a distal end, the outer tube is configured to extend away from a second end of the member at the proximal end and the intermediate portion is configured to extend away from the second end of the member to the distal end, the outer tube distal end is arranged to be flexed relative to the outer tube proximal end and the intermediate portion includes a bellows portion to accommodate such flexing, the intermediate portion is disposed between the outer tube distal end and the outer tube proximal end, wherein the second end of the member is opposingly disposed from the first end of the member and an entirety of the inner diameter of the outer tube is larger than the inner diameter of the inner tube; and
a septum mounted on the distal end of the outer tube, the septum is adapted to allow the catheter to be selectively inserted into, and removed from the patient's head through the site and maintain a gap between the catheter having a diameter smaller than the inner diameter of the outer tube to avoid contact between the outer tube and the catheter,
   whereby the catheter may be selectively inserted into, and removed from, the patient's head, reducing exposure of the catheter from the patient's scalp about the site.

In accordance with the present invention, there is further provided a catheter port adapted to be selectively secured topically to a patient's scalp about a site at which a catheter is to be selectively inserted into, and removed from of the patient's head, the catheter port including:
a cushion adapted to be secured to the patient's scalp about the site, the cushion having a bottom surface arranged to face toward the patient's scalp, an aperture through which the catheter is to be disposed and a top surface attached to a first end of a member;
an outer tube having an inner diameter, a proximal end, an intermediate portion and a distal end, the outer tube is configured to extend away from a second end of the member at the proximal end and the intermediate portion is configured to extend away from the second end of the member to the distal end, the outer tube distal end is arranged to be flexed relative to the outer tube proximal end and the intermediate portion includes a bellows portion to accommodate such flexing, the intermediate portion is disposed between the outer tube distal end and the outer tube proximal end, wherein the second end of the member is opposingly disposed from the first end of the member; and
a septum mounted on the distal end of the outer tube, the septum is adapted to allow the catheter to be selectively inserted into, and removed from the patient's head through the site and maintain a gap between the catheter having a diameter smaller than the inner diameter of the outer tube to avoid contact between the outer tube and the catheter,
whereby the catheter may be selectively inserted into, and removed from, the patient's head, reducing exposure of the catheter from the patient's scalp about the site.

In one embodiment, the septum includes a plurality of slits radiating outwardly from a common point to allow penetration of the catheter. In one embodiment, at least a portion of the cushion is provided with an adhesive coating that is adapted to secure the cushion to the patient's scalp. In one embodiment, at least a portion of the cushion is impregnated with an anti-microbial substance. In one embodiment, at least a portion of the cushion is impregnated with an antibiotic substance. In one embodiment, the catheter port further includes at least one of an antibiotic substance and an anti-microbial substance acting between the bottom surface of the cushion and the patient's scalp to provide a sterile field about the site. In one embodiment, the catheter port further includes a sleeve assembly having a proximal end and a distal end, the sleeve assembly including a sleeve adapter disposed on the proximal end of the sleeve assembly, a support block disposed on the distal end of the sleeve assembly and a sleeve interposed between the proximal end of the sleeve assembly and the distal end of the sleeve assembly, wherein the sleeve adapter is configured to be adaptable to the distal end of the outer tube and the catheter may be inserted through and removed through the sleeve adapter, sleeve and support block and protected within the sleeve.

In accordance with the present invention, there is provided a method of reducing exposure of a catheter from a patient's scalp about a site, the method including the steps of:

securing a cushion of a catheter port to the patient's scalp such that the cushion surrounds the site through which the catheter is to be disposed, the catheter port having a cushion and an outer tube, the cushion having a bottom surface, a top surface and an aperture, the bottom surface arranged to face toward the patient's scalp, the aperture disposed in the cushion and the top surface attached to a first end of a member, the outer tube having a proximal end, an intermediate portion and a distal end, the outer tube is configured to extend away from a second end of the member and the intermediate portion is configured to extend away from the second end of the member to the distal end, the outer tube distal end is arranged to be flexed relative to the outer tube proximal end and the intermediate portion includes a bellows portion to accommodate such flexing, the intermediate portion is disposed between the outer tube distal end and the outer tube proximal end, wherein the second end of the member is opposingly disposed from the first end of the member; and

disposing the catheter through the aperture of the cushion and an opening of the outer tube out of the catheter port such that exposure of the catheter from the patient's scalp about the site is reduced.

In one embodiment, the method further includes applying at least one of an antibiotic substance and an anti-microbial substance between a portion of the cushion and the patient's scalp to provide a sterile field about the site. In one embodiment, the cushion is formed of a foam material.

An object of the present invention is to provide a catheter port capable of reducing the exposure of a catheter from a patient's scalp when the catheter is being administered to the patient or when the catheter is left in place upon its administration.

An object of the present invention is to provide a catheter port capable of being secured to the scalp of a patient without fear of unintended detachment due to normal activities surrounding administration and maintenance of the catheter port.

An object of the present invention is to provide a catheter port capable of being administered without causing a significant amount of additional cost and labor over conventional catheter administering practices.

Whereas there may be many embodiments of the present invention, each embodiment may meet one or more of the foregoing recited objects in any combination. It is not intended that each embodiment will necessarily meet each objective. Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification.

### BRIEF DESCRIPTION OF THE DRAWINGS

In order that the manner in which the above-recited and other advantages and objects of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a top perspective view of one embodiment of a catheter port.

FIG. 2 is a cross-sectional view depicting a catheter port adapted to a catheter that is being inserted into human tissue.

FIG. 3 is a side view of one embodiment of a catheter port, depicting exemplary dimensions of the catheter port.

FIG. 4 is a side cross-sectional view of one embodiment of a catheter port.

FIG. 5 is a top view of one embodiment of a catheter port.

FIG. 6 is a bottom view of one embodiment of a catheter port.

FIG. 7 is a top perspective view of one embodiment of a catheter port adapted to a sleeve assembly.

FIG. 8 is a top perspective view of another embodiment of a catheter port.

FIG. 9 is a cross-sectional view of a catheter port adapted to a catheter.

FIG. 10 is a diagram depicting exemplary locations where catheter ports may be used on a human.

FIG. 11 is a diagram depicting exemplary locations where catheter ports may be used on a human.

FIGS. 12-19 are diagrams depicting exemplary steps taken to administer a catheter port.

FIG. 20 is a side cross-sectional view of one embodiment of a catheter port configured for use on a patient's head.

FIG. 21 is a side cross-sectional view of one embodiment of a catheter port configured for use on a patient's head.

FIG. 22 is a side cross-sectional view of one embodiment of a catheter port configured for use on a patient's head.

FIG. 23 is a cross-sectional view depicting a catheter port of FIG. 22 adapted to a catheter that is inserted into a patient's head.

FIG. 24 is a close-up view of the catheter port depicted in FIG. 23.

### PARTS LIST

2, 2A, 2B, 2C, 2D, 2E—catheter port
4—member
6—inner tube
8—flexible portion—bellow or pliable material
10—outer tube
12—septum
14—pin
16—catheter
18—tip of catheter
20—sleeve adapter
22—sleeve assembly
24—slot
26—direction in which thin wall portion bends
28—cushion
30—slits
32—thin wall portion
34—skin surface
36—skin layer
38—outer diameter of inner tube
40—outer diameter of member
42—outer diameter of cushion
44—thickness of member
46—thickness of cushion
48—inner diameter of outer tube
50—outer diameter of outer tube
52—length of inner tube
54—collar
56—height of collar
58—height of outer tube
60—inner diameter of inner tube
62—sleeve
64—distal end of sleeve
66—proximal end of sleeve
68—spacer
70—support block
72—patient
74—needle
76—puncture site
78—syringe
80—physician's hand
82—guidewire
84—guidewire cover
86—right shoulder
88—channel
90—needle tip
92—scalp
94—cranium
96—burr hole
98—incision
100—dura mater
102—ventricles
104—adhesive coating
106—adhesive coating
108—opening

### PARTICULAR ADVANTAGES OF THE INVENTION

The present catheter port eliminates any contact of catheters with skin or reducing the exposure of a catheter to scalp, thereby eliminating any transmission of diseases to a patient due to skin-catheter contact or reducing the opportunity for diseases to be transmitted from a patient's scalp to the patient. Without the present catheter port, any initial introduction or re-introduction of a catheter causes contamination of the segments of the catheter coming into contact with the skin or scalp. Any parts of a catheter that have come in contact with the skin or scalp can no longer be considered sterile despite present day attempts to create or re-create a sterile field on the skin surface with antimicrobial or antibiotic substances. The present catheter prevents any such contact from occurring.

As a catheter port provides isolation of the patient's epidermis with the insertion site of the patient, a catheter replacement can be performed in a sterile manner as an soiled catheter can be simply pulled out with the catheter port left intact at the insertion site and a new catheter inserted without coming into contact with the patient's epidermis. During the replacement of an old catheter with an unsoiled one, the epidermis surrounding the insertion site is most likely no longer sterile. By removing the possibility of any contact with the epidermis, an unsoiled catheter can be kept sterile during its insertion in the patient's body.

### DETAILED DESCRIPTION OF A PREFERRED EMBODIMENT

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

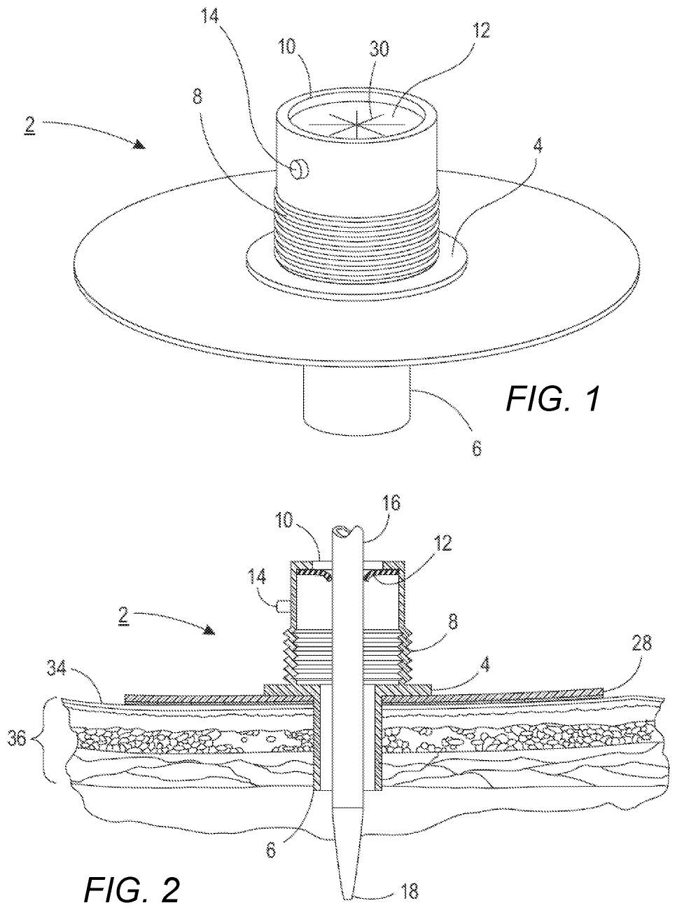

FIG. 1 is a top perspective view of one embodiment of a catheter port 2. FIG. 2 is a cross-sectional view depicting a catheter port 2 adapted to a catheter 16 that is being inserted into human tissue. The catheter port 2 is secured topically to a patient's body, e.g., via human skin layer 36, about a site at which a catheter 16 is to be inserted into the patient's body. At its basic form, the catheter port 2 includes a cushion 28 attached to a member 4 and an inner tube 6. The member 4 is essentially a flange serving as a platform to which a cushion 28 is attached in addition to adding rigidity to the catheter port 2. The member 4 includes an aperture through which a catheter 16 is to be inserted. The inner tube 6 includes one portion mounted on the member and another portion that is adapted to extend into the patient's body about the site for guiding movement of a catheter 16 relative to the site and for preventing any portion of the catheter from ever contacting the patient's epidermis. In one embodiment, the inner tube 6 is a cylindrical tube. In another embodiment, the inner tube is a frustoconical tube having a narrowing free end to make its insertion into a patient's body easier. In one embodiment, the inner tube 6 includes a smooth outer surface. In another embodiment, the inner tube 6 includes a ribbed surface to aid in preventing disengagement of the catheter port 2 from a patient's body. In one embodiment, the member 4 is a substantially circularly shaped flange.

In one embodiment, the catheter port 2 further includes an outer tube 10 and a septum 12. The outer tube 10 includes a proximal end, an intermediate portion and a distal end. The outer tube 10 is configured to be mounted to the member 4 at the proximal end and the intermediate portion is configured to extend away from the member 4 to the distal end. The septum 12 is mounted on the distal end of the outer tube 10 where it is arranged to create a gap between the outer tube 10 and the catheter 16 such that the catheter 16 is prevented from contacting engagement with the outer tube 10, further centering the catheter 16 within the inner tube 6, reducing any tendencies of the catheter 16 to be pressed against a corner of the catheter port 2. In the embodiment shown, the septum 12 includes a plurality of slits 30 radiating outwardly from a common point. In another embodiment, the septum includes a plate having an aperture to allow penetration of the catheter. The outer tube 10 distal end is arranged to be flexed relative to the outer tube 10 proximal end, facilitating any maneuvers that may be required of a catheter during use. The intermediate portion includes a flexible portion, constructed, e.g., from bellows portion 8 to accommodate such flexing, where the intermediate portion is disposed between the outer tube 10 distal end and the outer tube proximal end.

The site at which a catheter is to be inserted can be rather uneven due to the patient's skin contours or anatomical variations at the site. Therefore, a flat-faceted material may not be comfortably and sealingly disposed over such site. In one embodiment, a cushion 28 is configured to be disposed between the patient's epidermis about the site and the first surface of member 4 to accommodate differences in the distance between the patient's epidermis and the first surface of member 4. In this embodiment, the first surface of the member 4 is adhered or otherwise attached to a top surface of the cushion 28. In one embodiment, the cushion 28 is attached to the member 4 by an adhesive. In another embodiment, the cushion 28 is integrally built with the member 4 or fused to the member 4 via common welding techniques. In another embodiment, the cushion 28 is co-formed with the member 4 from a material, e.g., polyurethane, such that no welding or adhesives will be necessary to attach the cushion 28 to the member 4. An adhesive coating is provided to the bottom surface of the cushion 28 such that the cushion 28 may be secured to the patient's epidermis.

The cushion 28 is essentially a pliable disc with a hollowed center such that the inner tube 6 of a catheter port may be inserted through it. The cushion 28 is preferably constructed from a non-allergenic and malleable material (e.g., foam, etc.) suitable to conform to a patient's body structure at the application site of the catheter port 2. In one embodiment, at least a portion of the cushion 28 is impregnated with an anti-microbial substance to prevent, inhibit, or kill undesirable or harmful microbes. The presence of the anti-microbial substance can prevent or eliminate contamination of the surface of the equipment coming into contact with an undesirable or harmful microbe. In another embodiment, the cushion 28 is impregnated with an antibiotic substance.

In one embodiment, an antibiotic substance or an anti-microbial substance is applied where such substance acts between the patient-facing surface of the member 4 and the patient's epidermis 34 to provide a sterile field about the site.

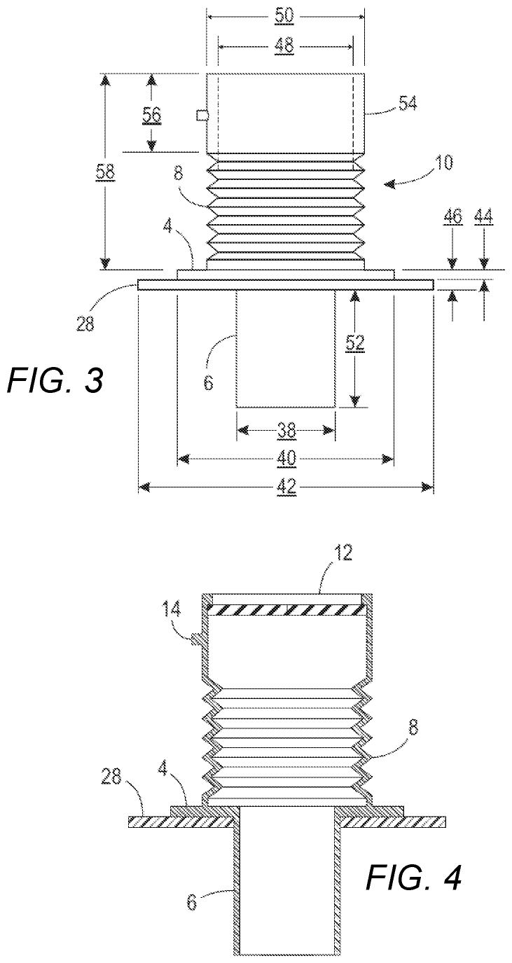

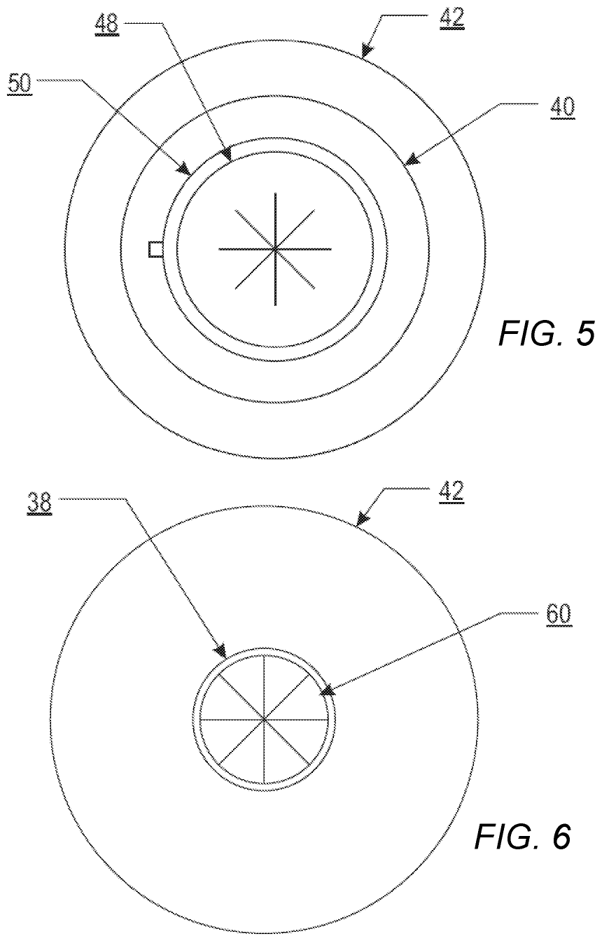

FIG. 3 is a side view of one embodiment of a catheter port 2, depicting exemplary dimensions of the catheter port. FIG. 4 is a side cross-sectional view of one embodiment of a catheter port. FIG. 5 is a top view of one embodiment of a catheter port 2. FIG. 6 is a bottom view of one embodiment of a catheter port 2. The cushions 28 are preferably configured significantly more prominently in diameter than the member 4 to provide sufficient cushioning of the catheter port 2 against the patient's skin 34 surface and to provide sufficient attachment surface area to reduce the likelihood of accidental detachment of the catheter port 2 from the patient while in use. The outer diameter 40 of the member 4 ranges from about 1 to about 2 cm. The outer diameter 42 of the cushion 28 ranges from about 2.5 cm to about 4.5 cm. The outer diameter 38 of inner tube ranges from about 4.5 mm to about 5 mm. The thickness 44 of the member 4 is about 2 mm. The thickness 46 of the cushion 28 is about 1 mm. The wall thickness 48 of the outer tube 10 is about 0.5 mm. The outer diameter 50 of the outer tube 10 is about 1 cm. The length 52 of inner tube 6 ranges from about 1 cm to about 3 cm. The outer tube 10 includes a collar 54. The height 56 of collar 54 is about 1.5 cm. The height 58 of outer tube 10 ranges from about 1 cm to about 2 cm. The wall thickness of the inner tube 6 is about 1 mm.

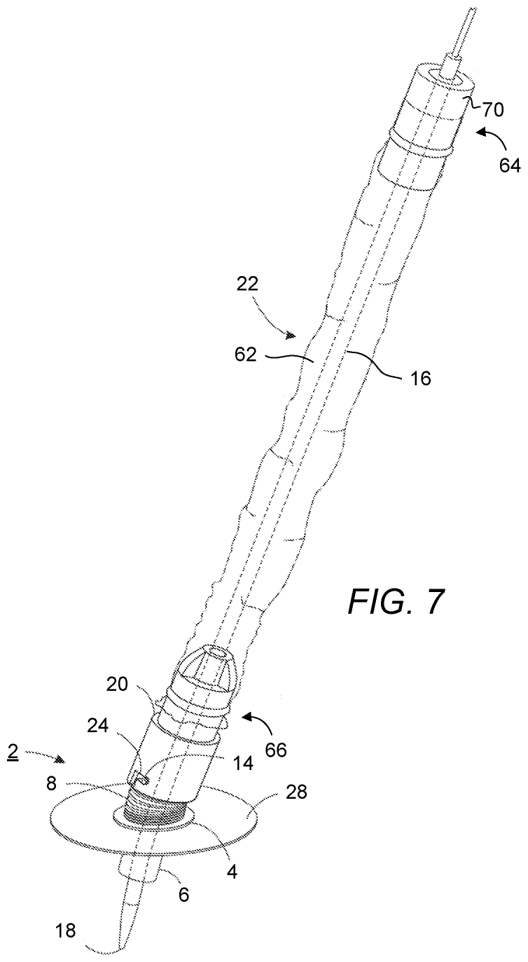

FIG. 7 is a top perspective view of one embodiment of a catheter port adapted to a sleeve assembly 22. Although the catheter port can be used without a sleeve assembly, when combined with the catheter port 2, the sleeve assembly 22 extends the length of the catheter port 2, increasing the amount of grasp points on the catheter port 2, facilitating the handling of the catheter port 2 relative to the catheter 16 and reducing the possibility of cross-contamination by protecting a larger area of the outer surfaces of the catheter. The sleeve assembly 22 has a proximal end 66 and a distal end 64. The sleeve assembly 22 includes a sleeve adapter 20 disposed on the proximal end 66 of the sleeve assembly 22, a support block 70 disposed on the distal end 64 of the sleeve assembly 22 and a sleeve 62 interposed between the proximal end 66 of the sleeve assembly 22 and the distal end 64 of the sleeve assembly 22. In use, the sleeve adapter 20 is configured to be capable of connection to the distal end of the outer tube 10. In one embodiment, the sleeve adapter 20 is equipped with a Bayonet Navy Connector (BNC) style connector which includes a slot 24. The slot 24 is configured to be coupled to a pin 14 disposed on the collar 54. In securing the sleeve assembly 22 to the catheter port 2, the sleeve adapter 20 is first brought close to the collar 54 such that they are roughly aligned and the opening of the slot 24 is aligned with the pin 14. The sleeve adapter 20 is advanced toward and over the outer tube 10 while continuing to be rotated until the pin 14 finally seats and is spring detented at the closed end of slot 24. Upon installation of the sleeve assembly 22, a catheter 16 may be inserted through the support block 70, sleeve 62 and sleeve adapter 20 into the catheter port 2. In operating environments where it is difficult to avoid contact of a catheter with the environment just outside of the surgical site, the sleeve assembly 22 reduces the amount of such contact while providing more grasping points for the surgical professionals so that no direct contact of the surgical professionals with the catheter 16 is necessary.

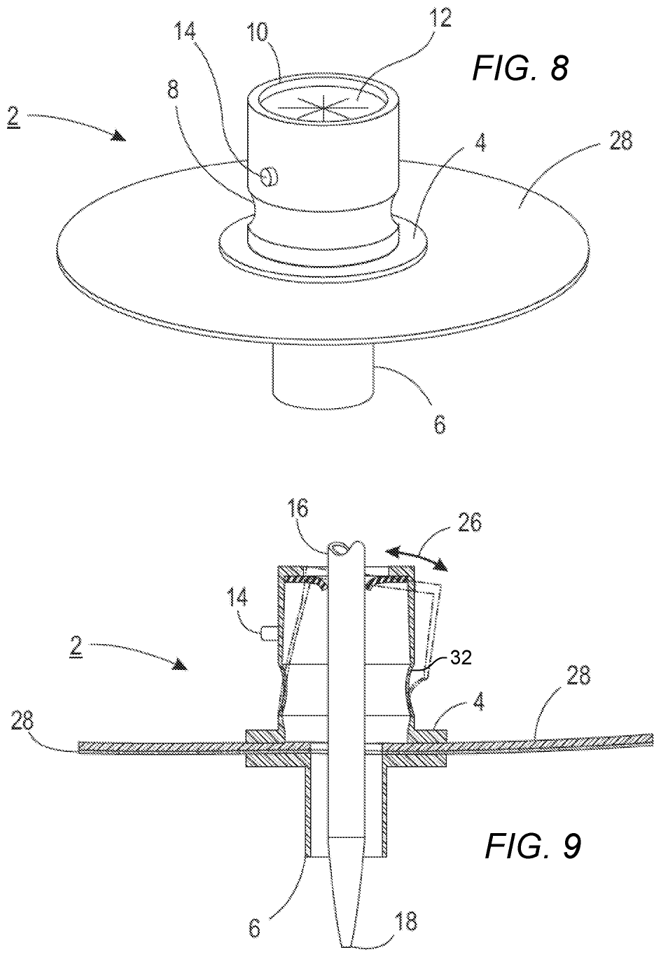

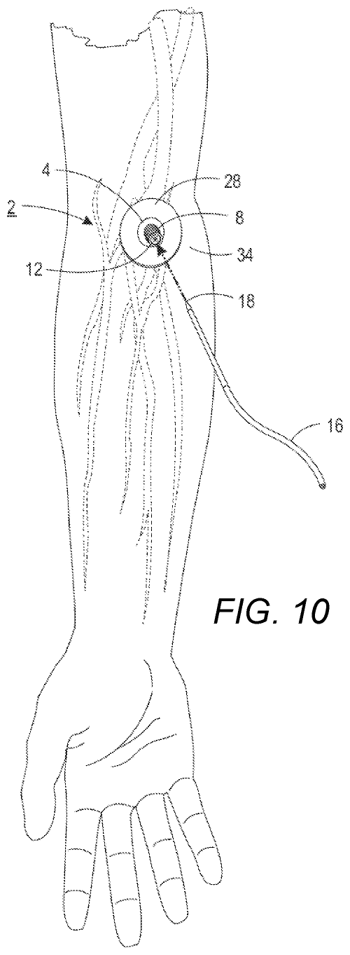

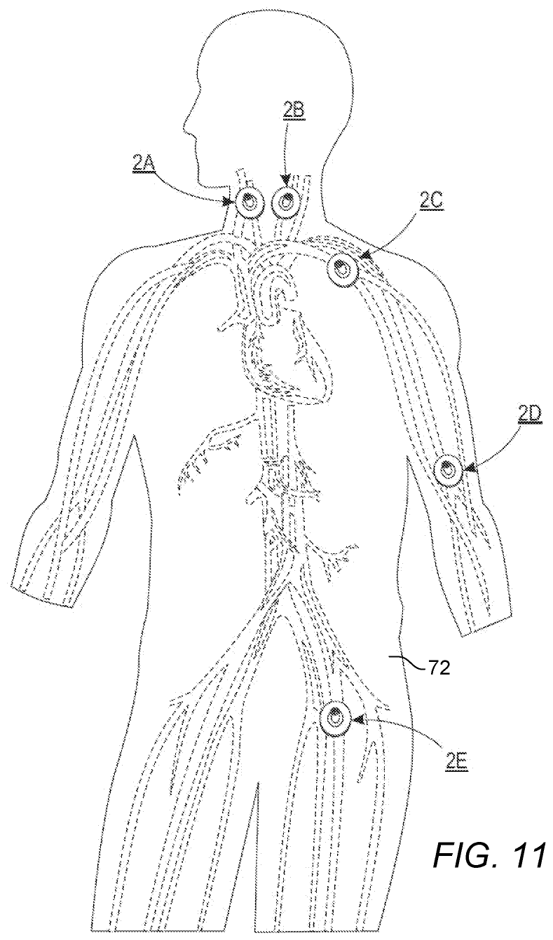

FIG. 8 is a top perspective view of another embodiment of a catheter port. FIG. 9 is a cross-sectional view of a catheter port adapted to a catheter. In this embodiment, the intermediate portion 8 includes a thin wall portion 32 to accommodate flexing. Again, the intermediate portion is disposed between the outer tube distal end and the outer tube proximal end. It shall be noted when a force is applied to the outer tube, e.g., when catheter is pulled against the septum, the outer tube bends in direction 26, accommodating such applied force while ensuring that the catheter 16 is still set a distance from the inner surfaces of the outer tube to keep such surfaces clean. FIGS. 10 and 11 are diagrams depicting exemplary locations where catheter ports may be used on a human. FIG. 10 depicts a Peripherally Inserted Central Catheter (PICC) administered through an entry point at a patient's arm. A PICC is inserted in a peripheral vein in the arm, such as the cephalic vein, basilic vein or brachial vein, and then advanced proximally toward the heart through increasingly larger veins, until the tip rests in the distal superior vena cava or cavoatrial junction. FIG. 11 depicts other access points to the central venous system. Catheter ports 2A, 2B are administered for access of catheters into to a large vein in the neck (internal jugular vein). Catheter port 2C is administered for access of catheters into the subclavian vein or axillary vein. Catheter port 2E is administered for access of catheters into the femoral vein in the groin. Although in conventional practices with catheters, extended periods of catheter access into the femoral vein in the groin is frowned upon as this area is typically moist and not hygienic. However, access through the groin presents a much lower health risk as compared to access through the internal jugular vein where catheter ports 2A and 2B are disposed. The present catheter port aids in lowering the risk of administering a catheter through the internal jugular vein by encouraging administration of a catheter through the groin instead although it can be used in with any central venous catheter.

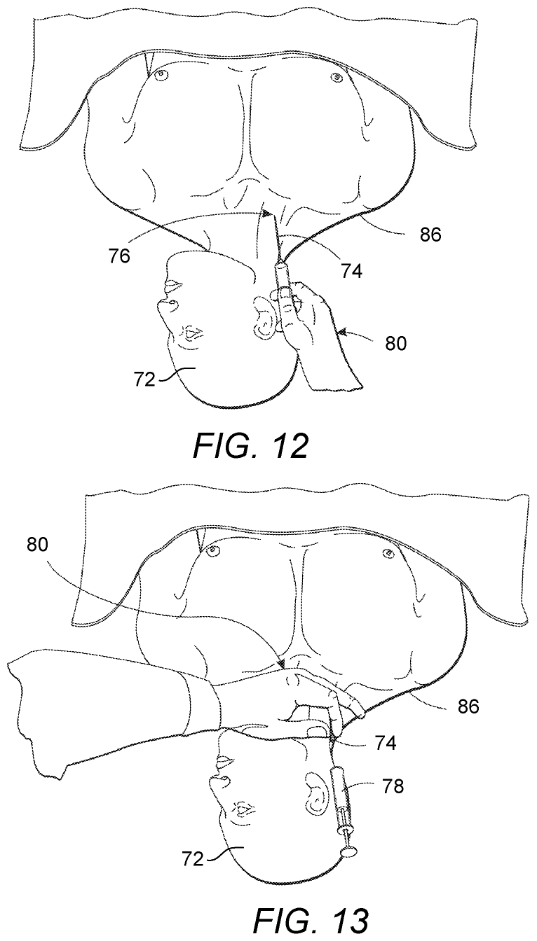

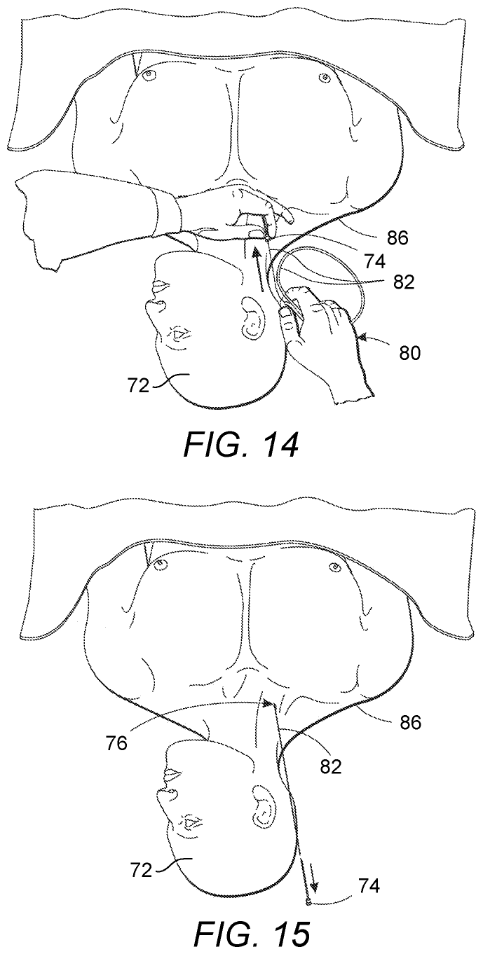

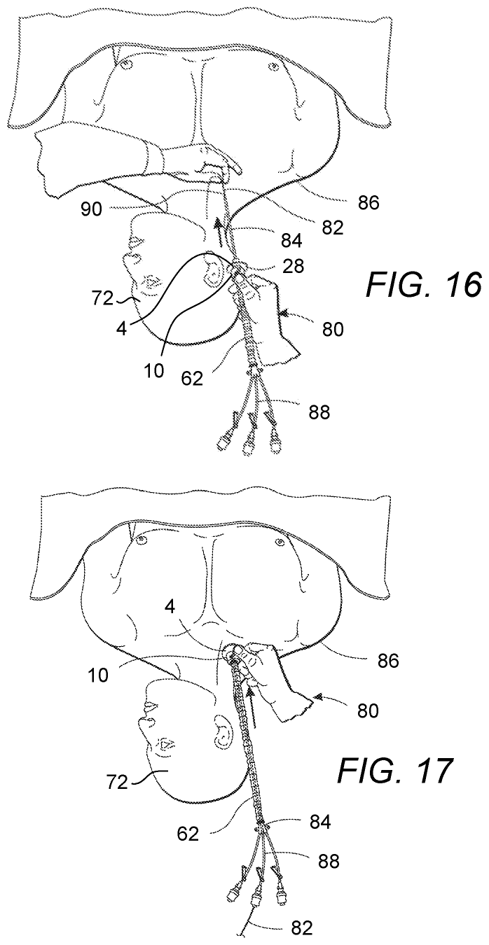

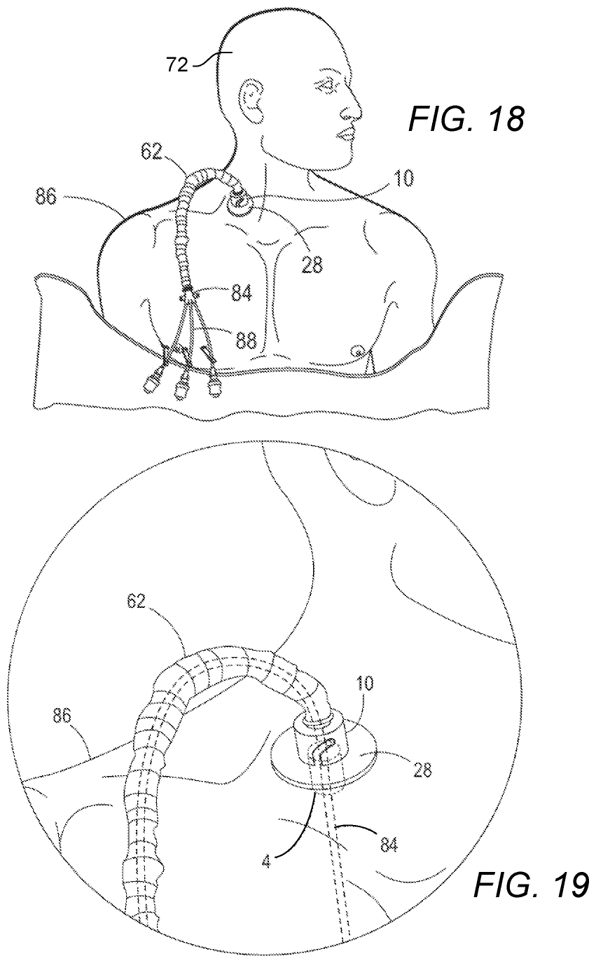

FIGS. 12-19 are diagrams depicting exemplary steps taken to administer a central venous catheter with a catheter port. In administering a catheter port, a sterile field is first created at the insertion or puncture site 76. A needle 74 is then placed through skin tissue into a body lumen, e.g., a vein until bleedback is achieved as shown in FIGS. 12-13. This is followed by introduction of a guidewire 82 through the skin and into the body lumen to a desired depth within the body lumen at its distal end as shown in FIGS. 14-15. The needle 74 is then exchanged for an introducer sheath with dilator, which are concentric tubes that are advanced over the guidewire 82 and into the body lumen. The needle 74 is removed by sliding the needle in a direction from the distal end of the guidewire 82 to the proximal end of the guidewire 82. The proximal end of the guidewire 82 is then inserted through a lumen of a dilator such that the dilator can be pulled in place to enlarge the passageway surrounding the needle 74. The dilator is then removed, and exchanged for a catheter. Prior to inserting a catheter in the patient's body, the catheter is inserted through a lumen of a present catheter port in the direction from the septum to the inner tube until the present catheter port is disposed roughly at the proximal end of the catheter and the guidewire 82 is removed. The catheter is then inserted in the patient's body through the insertion site 76 until its distal end is properly disposed. FIG. 16 depicts a catheter being inserted into the patient 72 with the aid of guidewire 82. FIG. 17 depicts a position of the catheter port having been seated in the surgical site. FIG. 18 depicts an administered catheter with a guidewire previously removed. FIG. 19 is a close-up view of an installed catheter port. Referring back to FIG. 16, a catheter port optionally coupled with a sleeve assembly is disposed over a multi-lumen catheter (a catheter having multiple dedicated catheter channels 88 via a single device) although a single channel catheter can also be used. The present catheter port is then pulled towards the insertion site 76 until the cushion becomes seated atop the skin or attached to the skin and the inner tube fully inserted in the patient's body/subcutaneous tissue. Therefore, it can be summarized that in administering a catheter port, the following steps shall be taken. First, a catheter port 2 is provided where the catheter port 2 includes a cushion 28 attached to a member 4, an inner tube 6 mounted to the member 4 and an aperture disposed in the member 4. A catheter is inserted through the catheter port 2 through the aperture of the member and the opening of the inner tube until the catheter port 2 is disposed at the proximal end of the catheter. The catheter is then inserted with its distal end first into the insertion site until the distal end has reached its intended depth or within the vein. The catheter port 2 is then pulled towards the patient until the cushion 28 has been placed with its bottom surface coming into contact with the patient's epidermis at the insertion site and the inner tube has extended into the patient's body about the site for guiding movement of a catheter 16 relative to the site and for preventing any portion of the catheter from ever contacting the patient's epidermis. The cushion 28 is adhered and secured to the patient's epidermis such that the cushion 28 surrounds the site through which the catheter is to be inserted. Upon installation of a catheter and a catheter port, no portion of the catheter will contact the patient's epidermis, thereby reducing the opportunity for infection due to contact with the patient's epidermis.

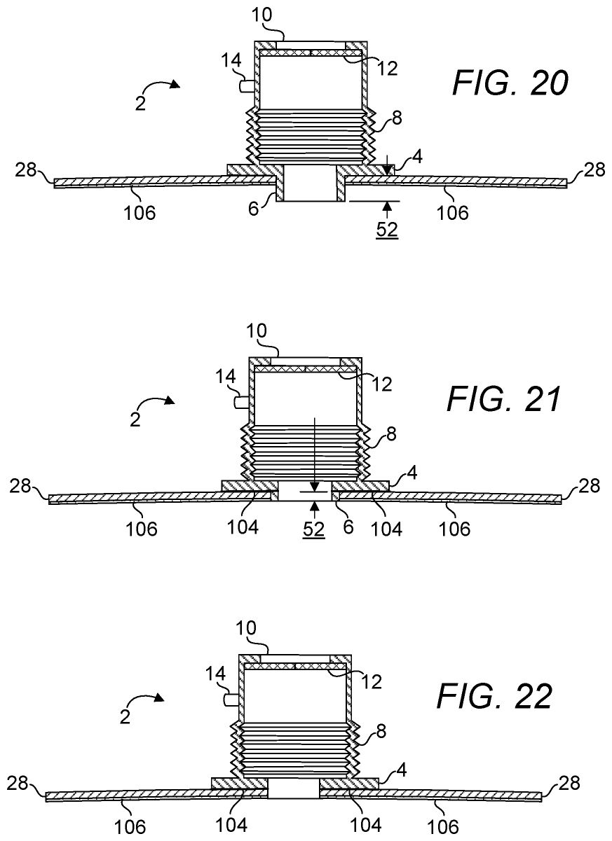

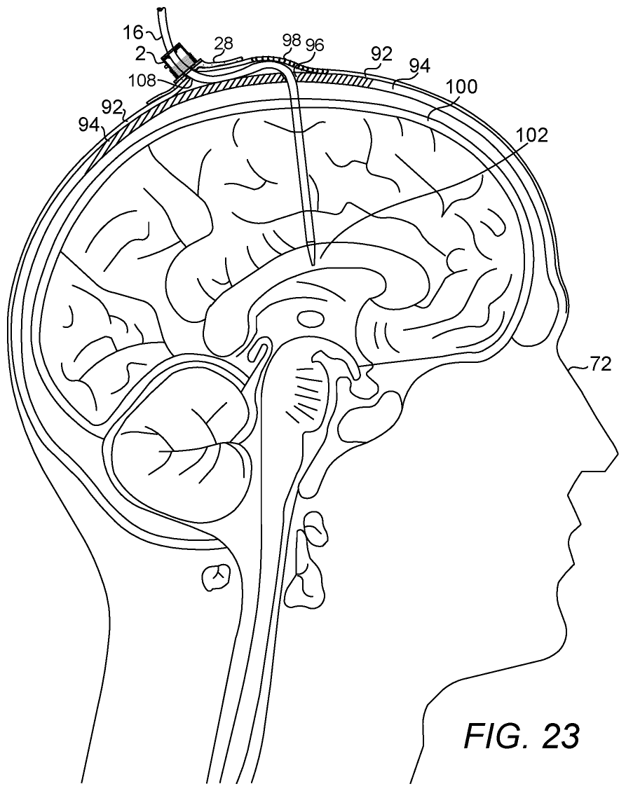

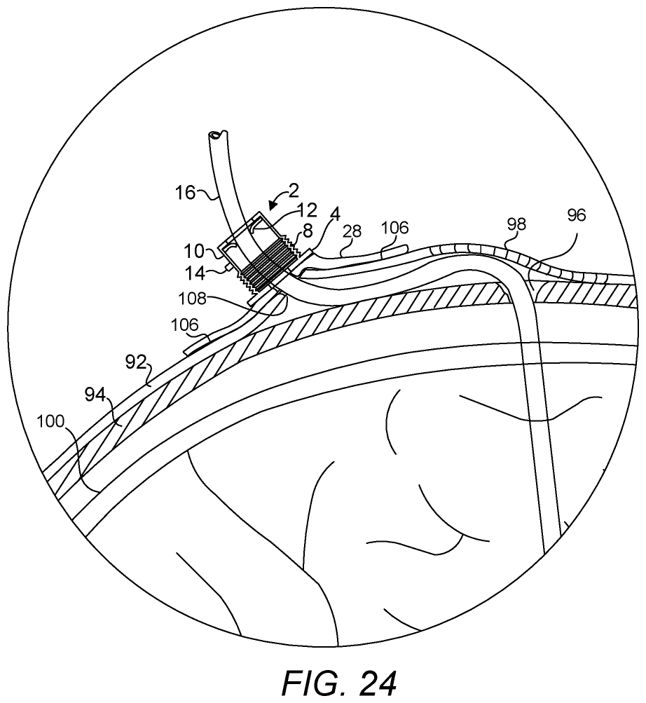

FIGS. 20-22 are side cross-sectional views of various embodiment of a catheter port 2 configured for use on a patient's head to alleviate hydrocephalus. FIG. 23 is a cross-sectional view depicting a catheter port of FIG. 22 adapted to a catheter that is inserted into a patient's head to alleviate hydrocephalus. FIG. 24 is a close-up view of the catheter port 2 depicted in FIG. 23. Hydrocephalus is a medical condition characterized by an excess accumulation of fluid in the brain. It results in increased intracranial pressure (ICP) and can cause severe brain damage or death if the pressure is not relieved. An external ventricular drain (EVD) is used to drain excess cerebrospinal fluid (CSF) in cases of temporary hydrocephalus such as brain hemorrhage and shunt malfunction in order to provide a therapeutic reduction in ICP.

Referring to FIGS. 20 and 21, in these embodiments, the catheter port 2 are similar to the embodiment disclosed in FIG. 4. However, the length 52 of inner tube 6 as shown in FIG. 20 is less than the length for a similar part shown in FIG. 4, i.e., the length of the inner tube 6 ranges from only about 1.0 mm to about 3 mm, or a length that is sufficient to surpass the thickness of the cushion 28 including adhesive coatings 104, 106. The length 52 of inner tube 6 as shown in FIG. 21 is yet less than the length of a similar part shown in FIG. 4, i.e., the length of the inner tube 6 ranges from about 0.5 mm to about 1.0 mm or just enough to extend to a depth at the same level as the cushion and adhesive coatings 104, 106 to avoid any protrusion of the inner tube 6 beyond the cushion 28 and the adhesive coating 106. Adhesive coating 104 is used to hold the cushion 28 firmly against member 4. The cushion 28 of the catheter port 2 of FIG. 20 need not be adhered to member 4 as the cushion 28 is sufficiently retained by friction by the inner tube 6 disposed through the aperture of the cushion 28.

In administering a catheter 16 to alleviate hydrocephalus, the CSF in the ventricles 102 must be drained. In order to reach the ventricles, a burr hole 96 is made in the cranium 94 and the dura mater 100 is punctured to allow the catheter 16 to be inserted in the head or brain. To reach the cranium 94 to create a burr hole 96, an incision 98 is first made in the scalp 92. A catheter 16 is then inserted through the burr hole 96 such that the distal end of the catheter 16 reaches the ventricles 102 for draining the CSF. A catheter port 2 is then disposed on the part of the scalp 92 through which the catheter 16 is to exit by attaching the catheter port 2 by its cushion 28 to the scalp 92 via adhesive coating 106. It shall be noted that this part of the scalp 92 is typically disinfected prior to the EVD procedure. The proximal end of the catheter 16 is then ready to be tunneled under the scalp 92 with the aid of a trocar to which the proximal end of the catheter is attached. Once an opening 108 in the scalp 92 has been formed, the catheter 16 is then routed through the aperture of the cushion 28 and through the catheter port 2. The proximal end of the catheter 16 is then capped off until further draining of the CSF is necessary and the catheter disposed extracorporeal is further secured to the scalp 92 at various spots of the scalp 92. All three embodiments shown in FIGS. 20-22 may be used. If FIG. 20 is used, the catheter port 2 may be disposed more securely over the opening in the scalp 92 produced using the trocar as the inner tube 6 is sufficiently long to line the opening. The embodiments of FIGS. 21 and 22 each offers a catheter port 2 that need not align perfectly with an opening 108 made with a trocar, making the catheter port 2 suitable for use with openings made with many makes and models of trocar. While being used in an EVD procedure, the present catheter port 2 reduces the exposure of a catheter disposed through the catheter port from a patient's scalp 92. In contrast to the application disclosed in FIG. 2, the opening in the scalp 92 through which a catheter 16 is disposed is made from under the scalp 92. As such, it is impossible to avoid contact of the catheter 16 with the scalp 92. Nevertheless, the area surrounding the opening is covered with the cushion 28 while the portion of catheter disposed extracorporeal at the opening is isolated from the scalp 92, reducing the potential for infection via the opening 108. Once the catheter 16 has been properly positioned, the incision 98 is sutured to seal off direct access to the burr hole 96. It shall be noted that the embodiments shown in FIGS. 20-23 may still be coupled with sleeves 62 shown in FIGS. 7, 18-19 to further shield at least a portion of a deployed catheter that is disposed outside of a patient's head and otherwise exposed to external contagions.

The detailed description refers to the accompanying drawings that show, by way of illustration, specific aspects and embodiments in which the present disclosed embodiments may be practiced. These embodiments are described in sufficient detail to enable those skilled in the art to practice aspects of the present invention. Other embodiments may be utilized, and changes may be made without departing from the scope of the disclosed embodiments. The various embodiments can be combined with one or more other embodiments to form new embodiments. The detailed description is, therefore, not to be taken in a limiting sense, and the scope of the present invention is defined only by the appended claims, with the full scope of equivalents to which they may be entitled. It will be appreciated by those of ordinary skill in the art that any arrangement that is calculated to achieve the same purpose may be substituted for the specific embodiments shown. This application is intended to cover any adaptations or variations of embodiments of the present invention. It is to be understood that the above description is intended to be illustrative, and not restrictive, and that the phraseology or terminology employed herein is for the purpose of description and not of limitation. Combinations of the above embodiments and other embodiments will be apparent to those of skill in the art upon studying the above description. The scope of the present disclosed embodiments includes any other applications in which embodiments of the above structures and fabrication methods are used. The scope of the embodiments should be determined with reference to the appended claims, along with the full scope of equivalents to which such claims are entitled.

What is claimed herein is:

1. A catheter port adapted to be selectively secured topically to a patient's scalp about a site at which a catheter is to be selectively inserted into, and removed from of the patient's head, said catheter port comprising:
a cushion adapted to be secured to the patient's scalp about the site, said cushion having a bottom surface arranged to face toward the patient's scalp, an aperture through which the catheter is to be disposed and a top surface attached to a first end of a member;
an inner tube having an inner diameter, a first end and a free second end, said inner tube mounted at said first end of said inner tube to said first end of said member, said free second end of said inner tube is adapted to only extend into the patient's head at and below the scalp about the site for guiding movement of the catheter relative to the site and for reducing exposure of the catheter from the patient's scalp;
an outer tube having an inner diameter, a proximal end, an intermediate portion and a distal end, said outer tube is configured to extend away from a second end of said member at said proximal end and said intermediate portion is configured to extend away from said second end of said member to said distal end, said outer tube distal end is arranged to be flexed relative to said outer tube proximal end and said intermediate portion comprises a bellows portion to accommodate such flexing, said intermediate portion is disposed between said outer tube distal end and said outer tube proximal end, wherein said second end of said member is opposingly disposed from said first end of said member and an entirety of said inner diameter of said outer tube is larger than said inner diameter of said inner tube; and
a septum mounted on said distal end of said outer tube, said septum is adapted to allow the catheter to be selectively inserted into, and removed from the patient's scalp through the site and maintain a gap between the catheter having a diameter smaller than said inner diameter of said outer tube to avoid contact between said outer tube and the catheter,
whereby the catheter may be selectively inserted into, and removed from, the patient's head, reducing exposure of the catheter from the patient's scalp about the site.

2. The catheter port as set forth in claim 1, wherein said septum comprises a plurality of slits radiating outwardly from a common point to allow penetration of the catheter.

3. The catheter port as set forth in claim 1, wherein at least a portion of said cushion is provided with an adhesive coating that is adapted to secure said cushion to the patient's scalp.

4. The catheter port as set forth in claim 1, wherein at least a portion of said cushion is impregnated with an anti-microbial substance.

5. The catheter port as set forth in claim 1, wherein at least a portion of said cushion is impregnated with an antibiotic substance.

6. The catheter port as set forth in claim 1, wherein said cushion is formed of a foam material.

7. The catheter port as set forth in claim 1, further comprising at least one of an antibiotic substance and an anti-microbial substance acting between said bottom surface of said cushion and the patient's scalp to provide a sterile field about the site.

8. The catheter port as set forth in claim 1, further comprising a sleeve assembly having a proximal end and a distal end, said sleeve assembly comprising a sleeve adapter disposed on said proximal end of said sleeve assembly, a support block disposed on said distal end of said sleeve assembly and a sleeve interposed between said proximal end of said sleeve assembly and said distal end of said sleeve assembly, wherein said sleeve adapter is configured to be adaptable to said distal end of said outer tube and the catheter may be inserted through and removed through said sleeve adapter, sleeve and support block and protected within said sleeve.

9. A catheter port adapted to be selectively secured topically to a patient's scalp about a site at which a catheter is to be selectively inserted into, and removed from of the patient's head, said catheter port comprising:
a cushion adapted to be secured to the patient's scalp about the site, said cushion having a bottom surface arranged to face toward the patient's scalp, an aperture through which the catheter is to be disposed and a top surface attached to a first end of a member;
an outer tube having an inner diameter, a proximal end, an intermediate portion and a distal end, said outer tube is configured to extend away from a second end of said member at said proximal end and said intermediate portion is configured to extend away from said second end of said member to said distal end, said outer tube distal end is arranged to be flexed relative to said outer tube proximal end and said intermediate portion comprises a bellows portion to accommodate such flexing, said intermediate portion is disposed between said outer tube distal end and said outer tube proximal end, wherein said second end of said member is opposingly disposed from said first end of said member; and
a septum mounted on said distal end of said outer tube, said septum is adapted to allow the catheter to be selectively inserted into, and removed from the patient's head through the site and maintain a gap between the catheter having a diameter smaller than said inner diameter of said outer tube to avoid contact between said outer tube and the catheter,
whereby the catheter may be selectively inserted into, and removed from, the patient's head, reducing exposure of the catheter from the patient's scalp about the site.

10. The catheter port as set forth in claim 9, wherein said septum comprises a plurality of slits radiating outwardly from a common point to allow penetration of the catheter.

11. The catheter port as set forth in claim 9, wherein at least a portion of said cushion is provided with an adhesive coating that is adapted to secure said cushion to the patient's scalp.

12. The catheter port as set forth in claim 9, wherein at least a portion of said cushion is impregnated with an anti-microbial substance.

13. The catheter port as set forth in claim 9, wherein at least a portion of said cushion is impregnated with an antibiotic substance.

14. The catheter port as set forth in claim 9, wherein said cushion is formed of a foam material.

15. The catheter port as set forth in claim 9, further comprising at least one of an antibiotic substance and an anti-microbial substance acting between said bottom surface of said cushion and the patient's scalp to provide a sterile field about the site.

16. The catheter port as set forth in claim 9, further comprising a sleeve assembly having a proximal end and a distal end, said sleeve assembly comprising a sleeve adapter disposed on said proximal end of said sleeve assembly, a support block disposed on said distal end of said sleeve assembly and a sleeve interposed between said proximal end of said sleeve assembly and said distal end of said sleeve assembly, wherein said sleeve adapter is configured to be adaptable to said distal end of said outer tube and the catheter may be inserted through and removed through said sleeve adapter, sleeve and support block and protected within said sleeve.

17. A method of reducing exposure of a catheter from a patient's scalp about a site, said method comprising the steps of:
securing a cushion of a catheter port to the patient's scalp such that said cushion surrounds the site through which the catheter is to be disposed, said catheter port having a cushion and an outer tube, said cushion having a bottom surface, a top surface and an aperture, said bottom surface arranged to face toward the patient's scalp, said aperture disposed in said cushion and said top surface attached to a first end of a member, said outer tube having a proximal end, an intermediate portion and a distal end, said outer tube is configured to extend away from a second end of said member and said intermediate portion is configured to extend away from said second end of said member to said distal end, said outer tube distal end is arranged to be flexed relative to said outer tube proximal end and said intermediate portion comprises a bellows portion to accommodate such flexing, said intermediate portion is disposed between said outer tube distal end and said outer tube proximal end, wherein said second end of said member is opposingly disposed from said first end of said member; and
disposing the catheter through said aperture of said cushion and an opening of said outer tube out of said catheter port such that exposure of the catheter from the patient's scalp about the site is reduced.

18. The method as set forth in claim 17, further comprising applying at least one of an antibiotic substance and an anti-microbial substance between a portion of said cushion and the patient's scalp to provide a sterile field about the site.

19. The method as set forth in claim 17, wherein said cushion is formed of a foam material.
